(19) United States
(12) Patent Application Publication
(10) Pub. No.: US 2018/0036086 A1
(43) Pub. Date: Feb. 8, 2018

(54) NOVEL ROBOTIC SURGICAL DEVICE

(71) Applicant: AMITH DEREK MENDONCA, BANGALORE (IN)

(72) Inventor: AMITH DEREK MENDONCA, BANGALORE (IN)

(21) Appl. No.: 15/290,284

(22) Filed: Oct. 11, 2016

(30) Foreign Application Priority Data
Aug. 3, 2016 (IN) 201641026508

(51) Int. Cl. A61B 34/30; A61B 34/20; A61B 17/02; A61B 17/16; A61B 17/14

(52) U.S. Cl. CPC A61B 34/30; A61B 17/1615; A61B 17/14; A61B 17/0218; A61B 17/1695; A61B 34/20; A61B 2017/1602; A61B 2034/302; A61B 2090/103

(57) ABSTRACT

A robotic surgical device configured to subcutaneously cut a bone of a vertebrate from a first location to a second location, wherein the robotic surgical device is introduced within the vertebrate through an incision made into the vertebrate.

### BACKGROUND

The present invention relates to robotic devices, and more particularly, to a robotic surgical device.

Today, autonomous and semi-autonomous robotic devices are used in various fields. The robotic devices are used to perform repetitive jobs or tasks which are considered dangerous or tedious for humans. Such robotic devices have become a part of many industrial and scientific areas like medicine, space exploration, construction, food packaging and are even used to perform complex surgeries.

With technological developments, robotic devices are now used to aid in complex surgeries. Such robotic devices help in providing minimally invasive surgical approaches and minimize human errors, by enabling actions which may be impossible to perform by a human. In some surgeries, the robotic devices are controlled via a computer control system by a surgeon who guides the robotic devices. In other surgeries, the surgeon uses a three-dimensional coordinate system to locate small targets inside a body to perform the surgery, which needs precise planning and execution. Such a method of performing guided surgery within the body is known as stereotactic method of performing the surgery. Stereotactic approaches are commonly employed in neurosurgical procedures and craniofacial surgery.

Craniofacial surgery is a subspecialisation of plastic surgery and maxillofacial surgery, that deals with the deformities of the head, skull, face, neck, jaws and associated structures. Craniofacial treatment often involves cutting and manipulation of craniofacial bones, in order to reconstruct bony deformities. Currently, such craniofacial treatment is conducted using long bi-coronal scalp incisions. Conventional craniofacial surgical methods are highly invasive and involve detaching a portion of the skull resulting in significant blood loss, dural damage and significant morbidity, with possible mortality.

Hence, there is a need for an improved system and method to address the aforementioned issues.

### BRIEF DESCRIPTION

In one embodiment, a robotic surgical device is provided. The robotic surgical device is configured to subcutaneously cut a bone of a vertebrate from a first location to a second location, wherein the robotic surgical device is introduced within the vertebrate through an incision made into the vertebrate.

In another embodiment, a method for cutting a bone in a vertebrate along a predetermined path from a first location to a second location is provided. The method includes controlling a robotic surgical device to subcutaneously reach a first bone cutting location in the predetermined path. The method also includes stabilising the robotic surgical device at the first bone cutting location. The method further includes using a bone cutting system located in the robotic surgical device to cut the bone subcutaneously at the first bone cutting location. The method also includes navigating the robotic surgical device to subcutaneously move a second bone cutting location along the predetermined path.

In yet another embodiment, a robotic surgical device configured to be introduced between a skin and a skull of a human being is provided. The robotic surgical device includes a bone cutting system configured to subcutaneously cut the skull of the human being from a first location to a second location along a predetermined path. The robotic surgical device also includes a movement system configured to subcutaneously move the robotic surgical device between the skin and the skull.

### BRIEF DESCRIPTION OF THE DRAWINGS

FIG. 1 is a schematic diagram representing a robotic surgical device placed in a vertebrate in accordance with an embodiment of the invention.

FIG. 2 is a schematic representation of a robotic surgical device used to cut a skull of a human being in accordance with an embodiment of the invention.

FIG. 3 is a schematic representation of a robotic surgical device configured to cut a bone in the vertebrate in accordance with an embodiment of the invention.

FIG. 4 is a schematic representation of a third robotic segment of FIG. 3 in accordance with an embodiment of the invention.

FIG. 5 is a schematic representation of a first robotic segment of FIG. 3 in accordance with an embodiment of the invention.

FIG. 6 is a schematic representation of a third robotic segment of FIG. 3 comprising a bone cutting system in accordance with an embodiment of the invention.

FIG. 7 is a flow chart representing steps involved in a method for cutting a bone in a vertebrate along a predetermined path from a first location to a second location in accordance with an embodiment of the invention.

### DETAILED DESCRIPTION

Embodiments of the present invention relate to a robotic surgical device configured to subcutaneously cut a bone of a vertebrate from a first location to a second location, wherein the robotic surgical device is introduced within the vertebrate through an incision made into the vertebrate.

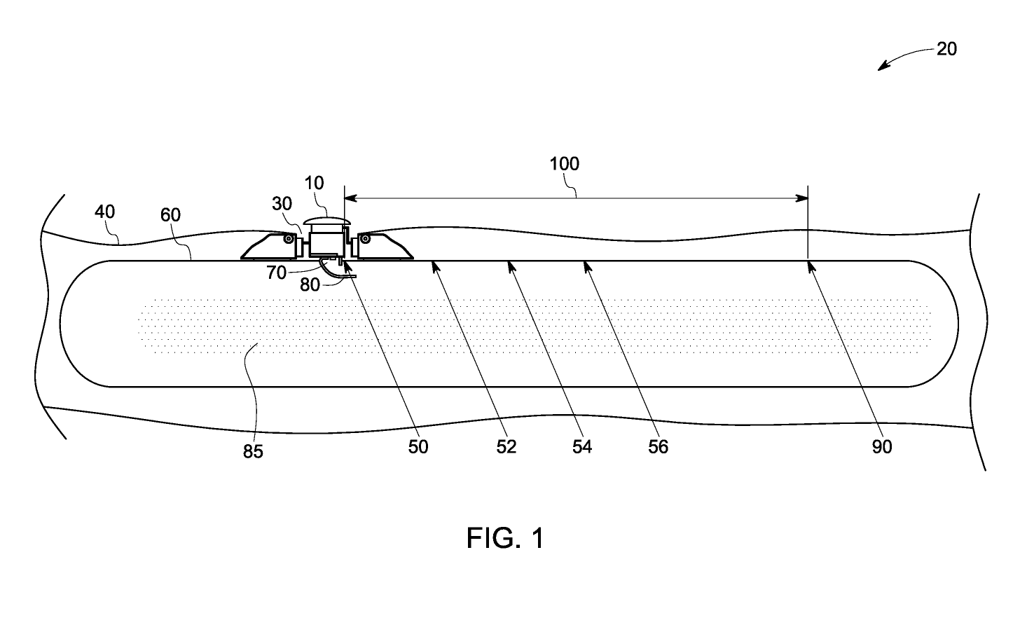

FIG. 1 is a schematic diagram representing a robotic surgical device 10 placed in a vertebrate 20 in accordance with an embodiment of the invention. In an event of conducting a bone surgery in the vertebrate 20, an incision 30 is made on a skin 40 of the vertebrate 20 at a first location 50. The robotic surgical device 10 is introduced between the skin 40 and a bone 60 of the vertebrate 20 at the first location 50 through the incision 30 made in the vertebrate 20. Furthermore, a cavity 70 is formed using a drilling mechanism in the bone 60 at the first location 50. In one embodiment, the robotic surgical device 10 may include a retractor 80 that is introduced below the bone 60 through the cavity 70 made at the first location 50 such that the retractor 80 is positioned between the bone 60 and a soft tissue 85 such as marrow within the bone 60. Subsequently, the robotic surgical device 10 is used to subcutaneously cut the bone 60 from the first location 50 to a second location 90 along a predetermined path 100. In one embodiment, the predetermined path may include a plurality of intermediate locations 52, 54, 56 between the first location 50 and the second location 90 at which the bone 60 is cut to enable cutting of the bone 60 along the predetermined path 100.

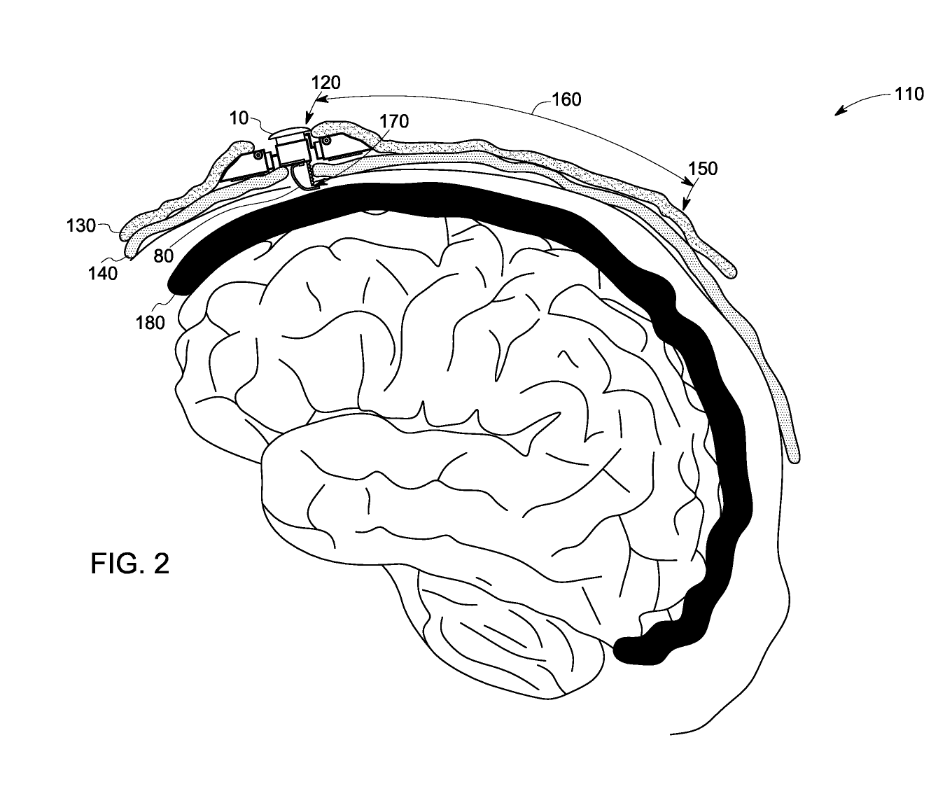

FIG. 2 is a schematic representation of a skull 110 of a human being comprising the robotic surgical device 10 for cutting the skull 110 in accordance with an embodiment of the invention. In embodiments, where the bone 60 is the skull 110 of the human being, the robotic surgical device 10 may be introduced in human being at a first location 120 in the skull 110 between a periosteum 130 and a loose sub-aponeurotic layer 140 at the first location 120 to cut the skull 110 from the first location 120 to a second location 150 on the skull 110 along a predetermined path 160. In embodiments where the robotic surgical device 10 includes the retractor 80, a cavity 170 may be drilled in the skull 110 at the first location 120 to insert the retractor 80 below the skull 110 to prevent damage to the duramater 180 during the cutting of the skull 110.

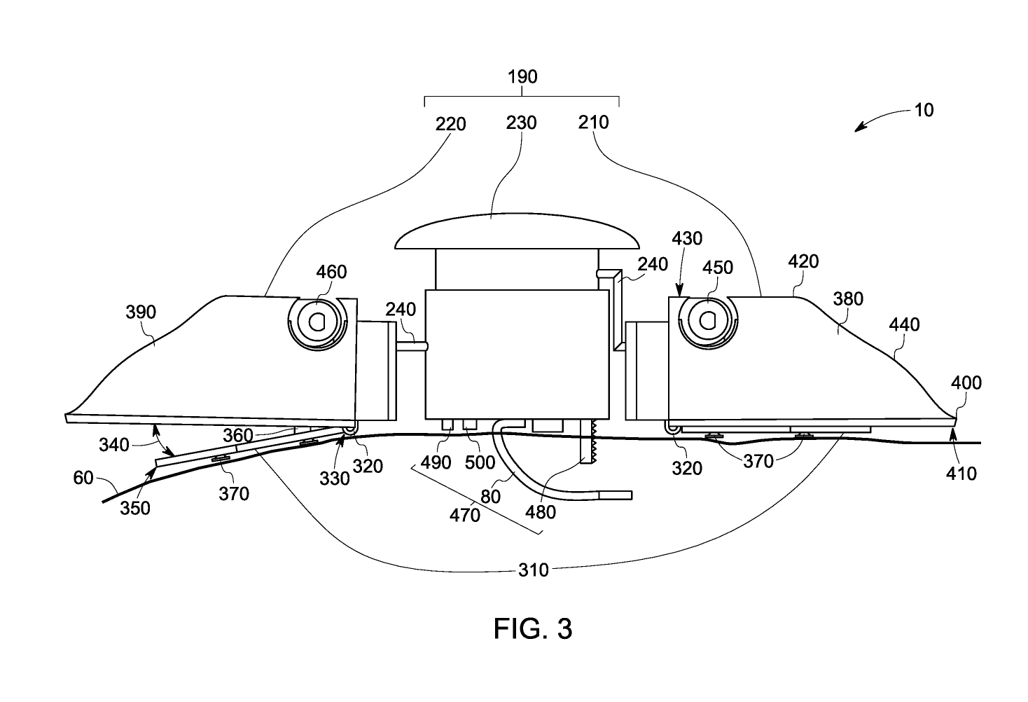

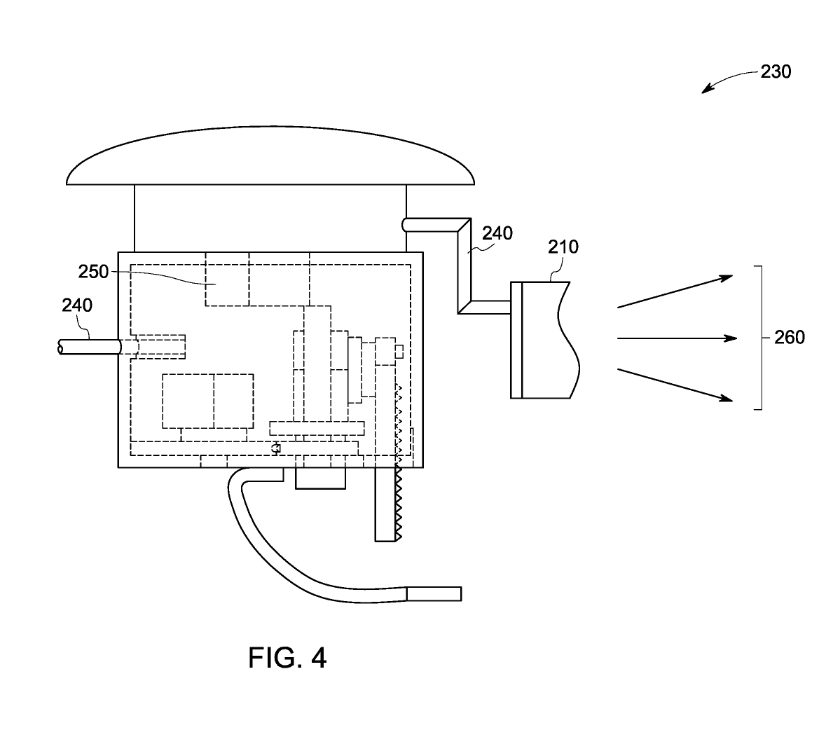

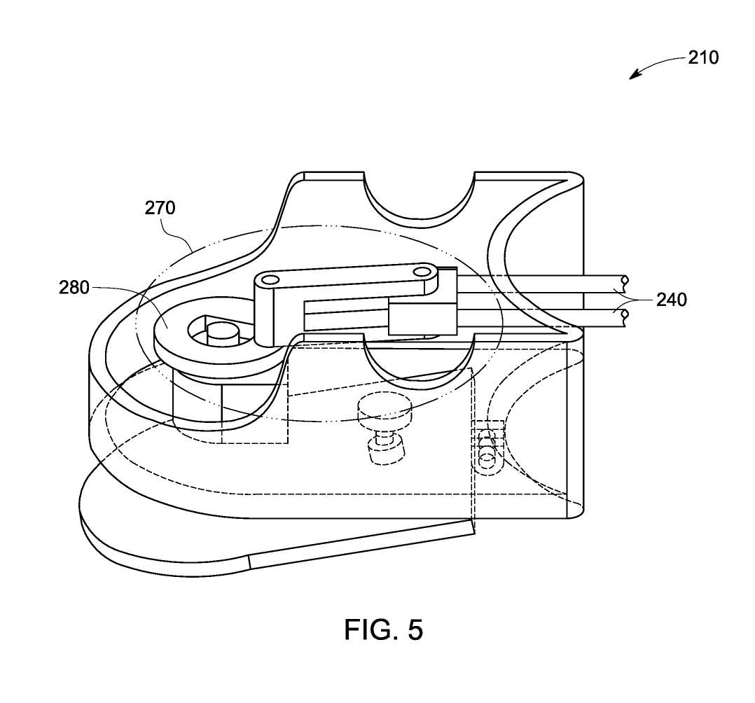

FIG. 3 is a schematic representation of the robotic surgical device 10 configured to cut the bone 60 in the vertebrate (FIG. 1) in accordance with an embodiment of the invention. The robotic surgical device 10 includes a movement system 190. In one embodiment, the movement system 190 includes a plurality of segments mechanically coupled to each other. In a specific embodiment, the movement system 190 includes a first robotic segment 210, a second robotic segment 220, and a third robotic segment 230 mechanically coupled to each other via a plurality of links 240. The movement system 190 is further explained in details with respect to FIG. 4-FIG. 5.

FIG. 4 is a schematic representation of the third robotic segment 230 of FIG. 3 in accordance with an embodiment of the invention. The movement system 190 includes a rotating joint 250 disposed in the third robotic segment 230. In one embodiment, the rotating joint 250 is a cylindrical joint. The cylindrical joint is mechanically coupled to the first robotic segment 210 via the plurality of links 240 to control a direction 260 of the first robotic segment 210. The rotating joint 250 is used to turn the first robotic segment 210 in the predetermined direction 260 that enables the robotic surgical device 10 to move the predetermined direction 260.

FIG. 5 is a schematic representation of the first robotic segment 210 of FIG. 3 in accordance with an embodiment of the invention. The first robotic segment 210 further includes a first slider crank system 270 mechanically coupled to the plurality of links 240 via a first connecting rod 280. The first slider crank system 270 enables the first robotic segment 210 to move in a forward direction and a backward direction to enable movement of the first robotic segment 210. Similarly, the second robotic segment 220 also includes a substantially similar second slider crank system (not shown) mechanically coupled to the plurality of links 240 via a second connecting rod (not shown).

Referring again to FIG. 3, the movement system 190 further includes a plurality of flaps 310. Each of the first robotic segment 210 and the second robotic segment 220 include a flap 310 for stabilizing the robotic surgical device 10 on the bone 60. Each of the flap 310 is mechanically coupled to the respective first robotic segment 210 and second robotic segment 220 via a pivot mechanism 320 at a first end 330 of the flap 310. The pivot mechanism 320 enables an angular movement 340 of a second end 350 of the flap 310 using a screw joint 360, where the angular movement 340 of the second end 350 may be defined as a vertical movement of the second end 350 of the flap 310 with respect to the first end 330 of the flap 310. The screw joint 360 is used to control the angular movement 340 of the second end 350 of the flap 310 based on a shape of the bone 60. For example, in situations, where the shape of the bone 60 is convex, the screw joint 360 may be actuated based on an interior angle of the bone 60 such that the angular movement 340 of the second end 350 of the flap 310 brings the flap 310 in contact with the bone 60. Furthermore, a plurality of suction caps 370 is provided below the flaps 310, which are actuated to affix the robotic surgical device 10 to the bone 60. In another embodiment, a plurality of tongs may be provided instead of suction caps 370 which can latch on to the bone 60 to affix the robotic surgical device 10 to the bone 60. Moreover, each of the flap 310 in the first robotic segment 210 and the second robotic segment 220 is configured to be controlled individually or in combination to affix the robotic surgical device 10 to the bone 60 based on the shape of the bone 60.

In operation, the movement system 190 is used to move the robotic surgical device 10 from the first location (FIG. 1) to an intermediate location along the predetermined path (FIG. 1). To this end, initially the flap 310 of the first robotic segment 210 is detached from the bone 60 by actuating the screw joint 360 to lift the flap 310. Subsequently, the first robotic segment 210 is moved forward using the first slider crank system 270 in the first robotic segment 210, which is actuated through a first motor (not shown) in the first robotic segment 210. Upon moving forward, the first robotic segment 210 is affixed to the intermediary location using the flap 310 of the first robotic segment 210. Furthermore, the second robotic segment 220 includes a second motor (not shown) that actuates the second slider crank system 290 in the second robotic segment 220 to push the third robotic segment 230 forward via the plurality of links 240. The first motor and the second motor operate simultaneously such that the first robotic segment 210 pulls the third robotic segment 230 via the plurality of links 240 and the second robotic segment 220 pushes the third robotic segment 230 forward via the plurality of links 240. Thus, the third robotic segment 230 moves forward and is subsequently attached to the bone 60. Later, the flap 310 of the second robotic segment 220 is lifted to detach from the bone 60 and the second robotic segment 220 moves forward using the second slider crank system 290 and attaches to the intermediate location using the flap 310 of the second robotic segment 220. The aforementioned process is repeated to move to a new intermediate location along the predetermined path. Such a configuration enables an earthworm like movement of the robotic surgical device 10 that also provides stability and reduces risk of errors during bone cutting.

Additionally, the first robotic segment 210 and the second robotic segment 220 include a first shell 380 and a second shell 390 respectively. The first shell 380 forms a covering for the first robotic segment 210 and is configured to separate the skin 40 from the bone 60 to enable movement of the robotic surgical device 10. To this end, a shape of the first shell 380 includes a sharp edge 400 at a front end 410 of the first robotic segment 210 and a flat surface 420 at a rear end 430 of the first robotic segment 210. Furthermore, an inclined surface 440 is used to connect the sharp edge 400 and the flat surface 420, which enables the first shell 380 to lift the skin 40 attached to the bone 60 and allowing the robotic surgical device 10 to move forward. Similarly, the second shell 390 forms a covering for the second robotic segment 220 and is of a shape substantially similar to the shape of the first shell 380. However, due to the positioning of the second shell 390 at the second robotic segment 220, the second shell 390 is configured to smoothly lay the skin 40 separated by the first shell 380 back on the bone 60 during the movement of the robotic surgical device 10. In one embodiment, each of the first shell 380 and the second shell 390 may be operatively coupled to a first shell actuator 450 and a second shell actuator 460 respectively. The first shell actuator 450 is used to exert additional force to separate the skin 40 from the bone 60 and the second shell actuator 460 is used to move the second shell 390 in a vertical direction to smoothly lay the skin 40 over the bone 60 during movement of the robotic surgical device 10.

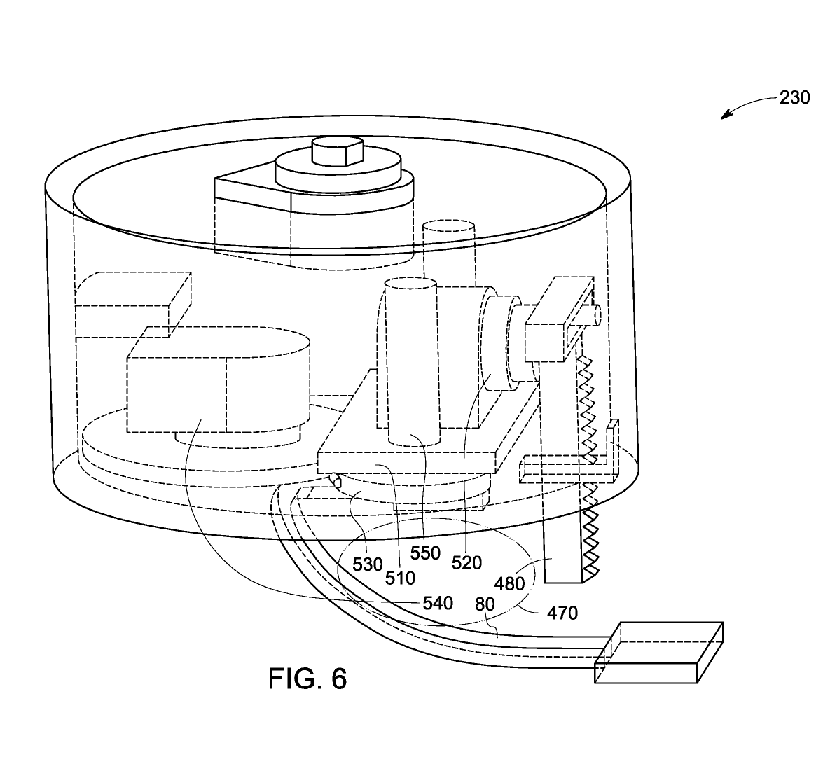

Furthermore, the robotic surgical device 10 includes a bone cutting system 470 provided in the third robotic segment 230. The bone cutting system 470 is used to cut the bone 60 at a location along the predetermined path (FIG. 1) such as the first location (FIG. 1), the intermediate location and the second location (FIG. 1). The bone cutting system 470 further includes a cutting means 480. In another embodiment, the bone cutting system 470 may include at least one of an irrigation system 490 and a suction system 500. The irrigation system 490 is used to irrigate the bone 60 at the location during the cutting of the bone 60. Furthermore, the suction system 500 is used to remove a bone residue after the bone 60 is cut at the location. Further details of the bone cutting system 470 are disclosed in FIG. 6.

FIG. 6 is a schematic representation of the third robotic segment 230 including the bone cutting system 470 located in the third robotic segment 230 in accordance with an embodiment of the invention. The bone cutting system 470 includes a cutting motor platform 510 on which a cutting motor 520 and the cutting means 480 are mounted. The cutting motor 520 is configured to drive the cutting means 480 using external power. In one embodiment, the cutting means 480 may include a saw blade. In a specific embodiment, the saw blade includes a T-joint that is used to attach the saw blade to a rotating disk of the cutting motor 520.

In another embodiment, the cutting means may include a milling burr. The cutting motor 520 and the milling burr are mounted on the cutting motor platform 510 such that an axle of the cutting motor 520 is placed facing towards the bone (FIG. 3) without the T-joint. In other embodiments, the cutting means 480 and the cutting motor 520 may be replaced with an ultrasonic bone cutting system, or a laser bone cutting system.

The bone cutting system 470 further includes the retractor 80. As previously discussed, the retractor 80 is inserted below the bone 60 through the cavity (FIG. 1) and is placed between the soft tissue (FIG. 1) such as the marrow and the bone 60. The retractor 80 prevents the cutting means 480 from damaging the soft tissue during the cutting of the bone 60. In the embodiment discussed in FIG. 2, where the skull is cut from the first location to the second location, the retractor 80 is located between the bone and the duramater. In such embodiments, the retractor 80 prevents damage to the duramater during cutting of the skull.

Furthermore, the bone cutting system 470 includes a height adjustable platform 530 configured to adjust a depth of the cut created by the cutting means 480. To this end, the height adjustable platform 530 includes a height adjustment motor 540 and a height adjustment screw joint 550. The height adjustment motor 540 actuates the height adjustment screw joint 550 to rotate in a clockwise or an anticlockwise direction to vertical move the cutting means 480. The vertical movement of the cutting means 480 increases or decreases the height of the cutting means 480 with respect to the bone 60 thereby altering the depth of the cut created by the cutting means 480.

With returning reference to FIG. 3, the robotic surgical device 10 also includes a conduit (not shown) operatively coupled to one or more external devices. The conduit includes at least one cable for enabling operations of one or more systems in the robotic surgical device 10. In one embodiment, the conduit may include an electrical cable for providing operating power to the robotic surgical device 10. In another embodiment, the conduit may include a data communication cable for transmitting and receiving data. In a specific embodiment, the data communication cable may transmit images obtained using an image capturing device in the robotic surgical device 10 to an external display. In another embodiment, the data communication cable may be used to provide control signal to the robotic surgical device 10 from an external controller. Furthermore, the data communication cable may be used to track a current location of the robotic surgical device 10. Based on the aforementioned uses of the data communication cable, a stereotactic navigation of the robotic surgical device 10 may be achieved for performing the bone cutting surgeries. In yet another embodiment, the conduit may also include an irrigation tube for providing a fluid to the irrigation system 490 from an external fluid source and a suction tube that enables the suction system 500 to remove the bone residue from the vertebrate (FIG. 1).

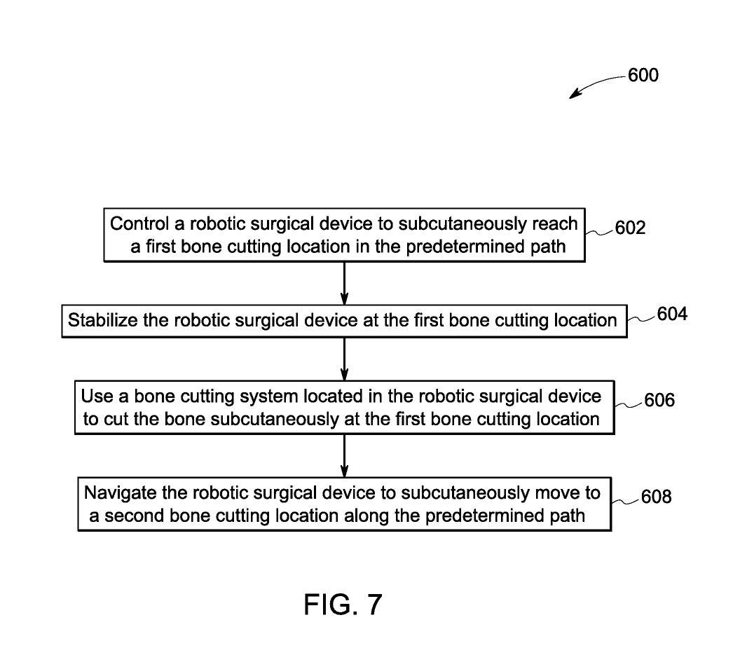

Furthermore, the operation of the robotic surgical device 10 is discussed with respect to FIG. 7 representing a flowchart depicting steps involved in a method 600 for cutting the bone 60 in the vertebrate 20 along the predetermined path 100 from the first location 50 to the second location 90 in accordance with an embodiment of the invention. It may be noted that the predetermined path 100 for cutting the bone 60 from the first location 50 to the second location 90 may include a plurality of bone cutting locations. The method 600 is discussed with respect to one instance, where the robotic surgical device 10 is used to cut the bone 60 at the first bone cutting location. The method 600 disclosed herein below may be repeated to cut the bone 60 at the plurality of bone cutting locations, thereby cutting the bone 60 from the first location 50 to the second location 90 along the predetermined path 100.

The method 600 includes controlling the robotic surgical device 10 to subcutaneously reach the first bone cutting location in the predetermined path in step 602. In one embodiment, the controlling the robotic surgical device 10 to subcutaneously reach the first bone cutting location in the predetermined path may include introducing the robotic surgical device 10 in the vertebrate 20 at the first location 50 through the incision 30 made in the vertebrate 20 at the first location 50. For the purpose of explanation of FIG. 7, the first location 50 and the first bone cutting location are the same locations as the robotic surgical device 10 is initially introduced in the vertebrate 20 at the first location 50. As previously discussed, the cavity 70 is drilled at the first bone cutting location 50 and the retractor 80 is inserted below the bone 60 such that the bone 60 is between the cutting means 480 and the retractor 80. In the embodiment of FIG. 2, where the skull 110 is cut from the first location 120 to the second location 150 along the predetermined path 160, the retractor 80 is inserted between the skull 110 and the duramater 180 to avoid damage to the duramater 180.

Subsequently, the robotic surgical device 10 is stabilized at the first bone cutting location 50 using the flaps 310 in step 604. In one embodiment, the flaps 310 of the first robotic segment 210 and the second robotic segment 220 of the robotic surgical device 10 are actuated using the screw joint 360 based on a curvature of the bone 60 to attach to the bone 60. In a specific embodiment, the flaps 310 affix to the bone 60 using a plurality of suction cups 370 or a plurality of tongs provided below the flaps 310.

The robotic surgical device 10 uses the bone cutting system 470 located in the robotic surgical device 10 to cut the bone 60 at the first bone cutting location 50 in step 606. In one embodiment, a height of a cutting means 480 is adjusted based on a thickness of the bone 60 to modify a depth of the cut in the bone 60. Furthermore, the cutting means 480 is actuated using a cutting motor 520, which allows the cutting means 480 to cut the bone 60. In another embodiment, the first bone cutting location 50 is irrigated using the irrigation system 490 during the bone cutting to clean the first bone cutting location 50. In yet another embodiment, a bone residue generated after cutting of the bone 60 is removed using the suction system 500 in the robotic surgical device 10.

Furthermore, the robotic surgical device 10 is navigated to subcutaneously move to a second bone cutting location 52 along the predetermined path 100 in step 608. It may be noted that the predetermined path 100 may include the plurality of intermediate locations 52, 54, 56 between the first location 50 and the second location 90 as previously discussed in FIG. 1 and one such intermediate location 52 is used as the second bone cutting location for the purposes of explanation of the method 600. In a specific embodiment, the robotic surgical device 10 is employs a stereotactic navigation for moving from the first bone cutting location 50 to the second bone cutting location 52. In one embodiment, the flap 310 of the first robotic segment 210 is detached from the bone 60 prior to moving to the second bone cutting location 52. Furthermore, the first robotic segment 210 moves forward to the second bone cutting location 52 from the first bone cutting location 50 using the first slider crank system 270 and attaches to the second bone cutting location 52 using the flap 310 of the first robotic segment 210. Subsequently, the flap 310 of the second robotic segment 220 detaches from the bone 60 and pushes the third robotic segment 230 forward to the second bone cutting location 52 using the second slider crank system 290 provided in the second robotic segment 220. Simultaneously, the first robotic segment 210 pulls the third robotic segment 230 to the second bone cutting location 52, which enables the movement of the second robotic segment 220 and the third robotic segment 230 to the second bone cutting location 52. The aforementioned process of cutting the bone 60 is repeated again to cut the bone 60 at the second bone cutting location 52. Similarly, the aforementioned method 600 may be repeated at the plurality of intermediate locations along the predetermined path 100 to cut the bone 60 from the first location 50 to the second location 90.

The various embodiments of the robotic surgical device described above enable a subcutaneous cutting of the bone, which leads to minimally invasive bone surgery, minimal incisions, minimal blood loss and minimal damage to the soft tissues within the bone. Also, the robotic surgical device minimizes damage to a duramater of the brain in craniofacial surgeries as the robotic surgical device includes a retractor that is placed between a skull bone and the duramater to prevent a cutting means from coming in contact with the duramater during the cutting of the bone.

It is to be understood that a skilled artisan will recognize the interchangeability of various features from different embodiments and that the various features described, as well as other known equivalents for each feature, may be mixed and matched by one of ordinary skill in this art to construct additional systems and techniques in accordance with principles of this disclosure. It is, therefore, to be understood that the appended claims are intended to cover all such modifications and changes as fall within the true spirit of the invention.

1. A robotic surgical device configured to subcutaneously cut a bone of a vertebrate from a first location to a second location, wherein the robotic surgical device is introduced within the vertebrate through an incision made into the vertebrate.

2. The robotic surgical device of claim 1, wherein the robotic surgical device comprises a first robotic segment, a second robotic segment and a third robotic segment.

3. The robotic surgical device of claim 2, wherein each of the first robotic segment and the second robotic segment comprises a flap for stabilizing the robotic surgical device on the bone.

4. The robotic surgical device of claim 3, wherein the flap comprises at least one of a plurality of suction caps or a plurality of tongs to affix the robotic surgical device to the bone.

5. The robotic surgical device of claim 2, wherein the first robotic segment and the second robotic segment comprise a first shell and a second shell respectively, and wherein the first shell is configured to separate a skin from the bone and the second shell is configured to lay a separated skin on the bone.

6. The robotic surgical device of claim 5, wherein the first robotic segment and the second robotic segment comprise a first shell actuator and a second shell actuator configured to lift the first shell and the second shell respectively.

7. The robotic surgical device of claim 2, wherein the third robotic segment comprises a bone cutting system.

8. The robotic surgical device of claim 7, wherein the bone cutting system comprises cutting means, wherein the cutting means comprises a saw blade or a milling burr.

9. The robotic surgical device of claim 7, wherein the bone cutting system comprises a height adjustable platform for adjusting a depth of a cut created using the cutting means.

10. The robotic surgical device of claim 2, wherein the third robotic segment comprises a retractor introduced through a cavity made in the bone at the first location, wherein the bone is between a bone cutting system and the retractor.

11. The robotic surgical device of claim 2, wherein each of the first robotic segment and the second robotic segment comprises a slider crank system for moving the robotic surgical device.

12. The robotic surgical device of claim 2, wherein the third robotic segment comprises a cylindrical joint mechanically coupled to the first robotic segment via a plurality of links for controlling a direction of the robotic surgical device.

13. The robotic surgical device of claim 1, wherein the robotic surgical device is configured to cut a skull.

14. A method for cutting a bone in a vertebrate along a predetermined path from a first location to a second location, comprising:
controlling a robotic surgical device to subcutaneously reach a first bone cutting location in the predetermined path;
stabilising the robotic surgical device at the first bone cutting location;
using a bone cutting system located in the robotic surgical device to cut the bone subcutaneously at the first bone cutting location; and
navigating the robotic surgical device to subcutaneously move to a second bone cutting location along the predetermined path.

15. The method of claim 14, wherein controlling the robotic surgical device to subcutaneously reach the first bone cutting location in the predetermined path further comprises introducing the robotic surgical device in the vertebrate at the first bone cutting location through an incision made in the vertebrate at the first bone cutting location.

16. The method of claim 15, further comprising forming a cavity at the first bone cutting location and introducing a retractor of the robotic surgical device through the cavity such that the bone is placed between a cutting means and the retractor.

17. The method of claim 14, wherein navigating the robotic surgical device to the second bone cutting location comprises using stereotactic navigation for navigating the robotic surgical device.

18. The method of claim 14, further comprising adjusting a height of the bone cutting system based on a thickness of the bone at the first bone cutting location.

19. A robotic surgical device configured to be introduced between a skin and a skull of a human being comprising:
a bone cutting system configured to subcutaneously cut the skull of the human being from a first location to a second location along a predetermined path; and
a movement system configured to subcutaneously move the robotic surgical device between the skin and the skull.

20. The system of claim 19, wherein the movement system comprises a plurality of robotic segments mechanically coupled to each other through a slider crank system.
